(19) United States
(12) Patent Application Publication
(10) Pub. No.: US 2011/0172421 A1
(43) Pub. Date: Jul. 14, 2011

(54) N-(alpha-AROMATIC GROUP-SUBSTITUTED-2-NITRO-4,5-DIALKOXYBENZYLOXYCARBONYL)AMINE COMPOUND AND PROCESS FOR PRODUCING THE SAME

(75) Inventors: Isao Yamagami, Minato-ku (JP); Yoshihiko Maeda, Minato-ku (JP); Hiroshi Yasuda, Minato-ku (JP); Katsumi Murofushi, Minato-ku (JP)

(73) Assignee: SHOWA DENKO K.K., Minato-ku, Tokyo (JP)

(21) Appl. No.: 13/121,048

(22) PCT Filed: Oct. 1, 2009
(86) PCT No.: PCT/JP2009/067159

§ 371 Date: Mar. 25, 2011

(30) Foreign Application Priority Data
Oct. 2, 2008 (JP) 2008-257159

(51) Int. Cl. C07D 265/30; C07C 269/00; C07D 211/06; C07C 271/06

(52) U.S. Cl. 544/167; 560/157; 546/226

(57) ABSTRACT

An object of the present invention is to provide a novel photobase generator which can sensitively generate a base even by h-ray in place of a conventional 2-nitro-4,5-dimethoxybenzyloxycarbonylamine compound. Disclosed is an N-(α-aromatic group-substituted-2-nitro-4,5-dialkoxybenzyloxycarbonyl)amine compound represented by the following general formula (I).

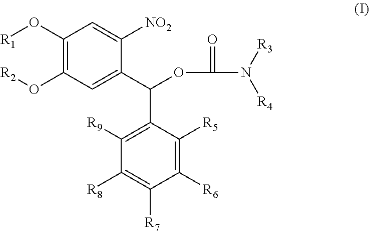

(In the above formula (I), R¹ to R⁹ denote specific groups.)

### TECHNICAL FIELD

The present invention relates to a novel compound useful as a photobase generator and the like, an N-(α-aromatic group-substituted-2-nitro-4,5-dialkoxybenzyloxycarbonyl)amine compound and a process for producing the same. The N-(α-aromatic group-substituted-2-nitro-4,5-dialkoxybenzyloxycarbonyl)amine compound according to the present invention is a compound which gives a response to ultraviolet with a specific wavelength to be decomposed and generate a base.

### BACKGROUND ART

In recent years, attention has been focused on a compound which is decomposed by radiation such as ultraviolet to generate a base (a photobase generator). The generated base, such as an amine compound, functions as a catalyst for crosslinking reaction and polymerization reaction or as a crosslinking agent itself. The photobase generator has been used particularly for use in photoresist.

In the photoresist-related field, as a ray employed in pattern formation, so-called i-ray, which is ultraviolet with a wavelength of approximately 365 nm, has been employed. It is therefore desired that the photobase generator employed in the photoresist should be a compound which has an absorption peak in a wavelength region of approximately 365 nm and efficiently generates an amine compound. In recent years, moreover, ultraviolet with a wavelength of approximately 405 nm, which is called h-ray, has been employed in pattern formation and thus there is a demand for the development of a photobase generator having an absorption peak in these wavelength regions.

For example, Non-Patent Document 1 discloses, as a photobase generator, 2-nitrobenzyloxycarbonylcyclohexylamine represented by the following formula.

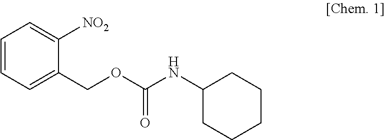

This compound, however, fails to have sensitivity to h-ray; and therefore it is difficult to generate a base by applying h-ray to this compound.

Patent Document 1 discloses a photobase generator having two or more groups in the molecule, the group being represented by the following formula.

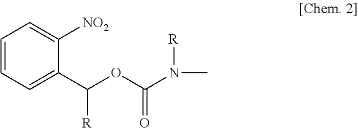

In the above formula, R is hydrogen, an alkyl group or an aryl group.

The disclosure of Patent Document 1, however, is only about the generation of a base in employing an ultrahigh-pressure mercury lamp (the wavelength is primarily between 280 and 600 nm). Whether or not the photobase generator disclosed in Patent Document 1 will generate a base by i-ray or h-ray is not clearly described in Patent Document 1.

Patent Document 2 discloses a photobase generator represented by the following formula.

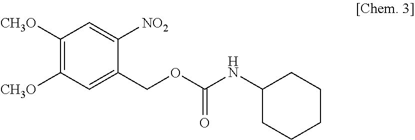

2-nitro-4,5-dimethoxybenzyloxycarbonylcyclohexylamine disclosed in Patent Document 2, however, is not described to have sensitivity to h-ray, although it has sensitivity to i-ray.

### CITATION LIST

### Patent Document

Patent Document 1: JP-B-S51-46159
Patent Document 2: JP-A-H06-345711
Non-Patent Document
Non-Patent Document 1: J. Am. Chem. Soc., 113, 4303-4313 (1991)

### SUMMARY OF THE INVENTION

### Problem to be Solved by the Invention

It is an object of the present invention to provide a novel photobase generator which can sensitively generate a base even by h-ray in place of a conventional 2-nitro-4,5-dimethoxybenzyloxycarbonylamine compound. Further, it is another object of the present invention to provide a simplified process for producing the novel photobase generator.

The present inventors studied to solve these problems and has developed as a novel photobase generator, specific N-(α-aromatic group-substituted-2-nitro-4,5-dialkoxybenzyloxycarbonyl)amine compounds and a simplified production process thereof.

That is, the gist of the present invention is as follows.

[1] An N-(α-aromatic group-substituted-2-nitro-4,5-dialkoxybenzyloxycarbonyl)amine compound represented by the following formula (I).

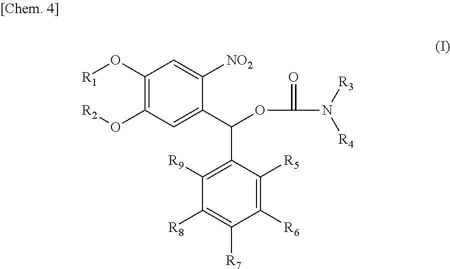

In the general formula (I), R₁ and R₂ are each independently an alkyl group having 1 to 12 carbon atoms which may have a substituent group or an aryl group having 6 to 12 carbon atoms which may have a substituent group, and R₁ and R₂ may be bonded to form an alkylene group having 1 to 12 carbon atoms which may have a substituent group or an arylene group having 6 to 12 carbon atoms which may have a substituent group;

R₃ and R₄ are each independently a hydrogen atom, an alkyl group having 1 to 12 carbon atoms which may have a substituent group or an aryl group having 6 to 12 carbon atoms which may have a substituent group, at least one of R₃ and R₄ is not a hydrogen atom, and R₃ and R₄ may be bonded to form a cyclic structure which may contain a hetero atom; and

R₅ to R₉ are each independently a hydrogen atom, an alkyl group having 1 to 12 carbon atoms, an aryl group having 6 to 12 carbon atoms, an alkoxy group having 1 to 12 carbon atoms, a halogen atom, a cyano group, an amino group, an alkylamino group having 1 to 12 carbon atoms, an acyloxy group having 1 to 12 carbon atoms, a nitro group or an acyl group having 1 to 12 carbon atoms.

[2] The N-(α-aromatic group-substituted-2-nitro-4,5-dialkoxybenzyloxycarbonyl)amine compound as described in the above [1], wherein in the general formula (I), R₃ is a hydrogen atom.

[3] The N-(α-aromatic group-substituted-2-nitro-4,5-dialkoxybenzyloxycarbonyl)amine compound as described in the above [1], wherein in the general formula (I), R₁ and R₂ are methyl groups, R₃ and R₄ are bonded to form a morpholyl group and R₅ to R₉ are all hydrogen atoms.

[4] The N-(α-aromatic group-substituted-2-nitro-4,5-dialkoxybenzyloxycarbonyl)amine compound as described in the above [1] or [2], wherein in the general formula (I), R₁ and R₂ are methyl groups, R₃ is a hydrogen atom, R₄ is a cyclohexyl group and R₅ to R₉ are all hydrogen atoms.

[5] The N-(α-aromatic group-substituted-2-nitro-4,5-dialkoxybenzyloxycarbonyl)amine compound as described in the above [1], wherein the compound represented by the general formula (I) is at least one compound selected from the group consisting of
N-(α-phenyl-2-nitro-4,5-dimethoxybenzyloxycarbonyl)-2,6-dimethylpiperidine,
N-(α-(4-nitrophenyl)-2-nitro-4,5-dimethoxybenzyloxycarbonyl)-2,6-dimethylpiperidine,
N-(α-(2-nitrophenyl)-2-nitro-4,5-dimethoxybenzyloxycarbonyl)-2,6-dimethylpiperidine,
N-(α-(2-nitro-4,5-dimethoxyphenyl)-2-nitro-4,5-dimethoxybenzyloxycarbonyl)-2,6-dimethylpiperidine,
N-(α-phenyl-2-nitro-4,5-dimethoxybenzyloxycarbonyl)-piperidine and
N-(α-(2-nitro-4,5-dimethoxyphenyl)-2-nitro-4,5-dimethoxybenzyloxycarbonyl)-piperidine.

[6] A process for producing the N-(α-aromatic group-substituted-2-nitro-4,5-dialkoxybenzyloxycarbonyl) amine compound as described in the above [1], comprising reacting an aldehyde compound represented by the following general formula (II) and an aromatic compound represented by the following general formula (III) and reacting the compound obtained by the reaction with a compound represented by the following general formula (IV).

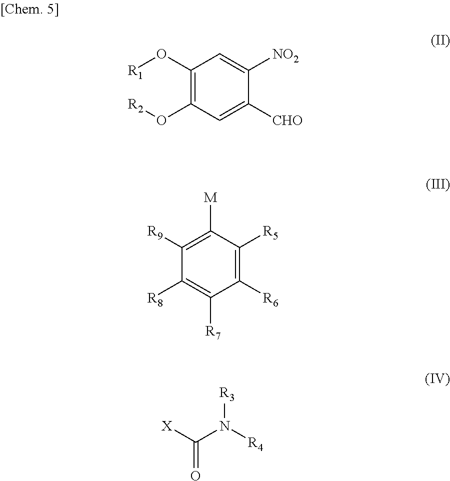

In the general formula (II), R₁ and R₂ are each the same as R₁ and R₂ in the general formula (I);

in the general formula (III), R₅ to R₉ are each the same as R₅ to R₉ in the general formula (I), M is a substituent group containing a metal, and the metal is Mg, Zn, Li, Sn or Cu; and

in the general formula (IV), R₃ and R₄ are each the same as R₃ and R₄ in the general formula (I), and X is a halogen atom selected from a fluorine atom, a chlorine atom, a bromine atom and an iodine atom.

[7] A process for producing the N-(α-aromatic group-substituted-2-nitro-4,5-dialkoxybenzyloxycarbonyl)amine compound as described in the above [2], comprising reacting an aldehyde compound represented by the following general formula (II) with an aromatic compound represented by the following general formula (III) and reacting the compound obtained by the reaction with an isocyanate compound represented by the following general formula (V):

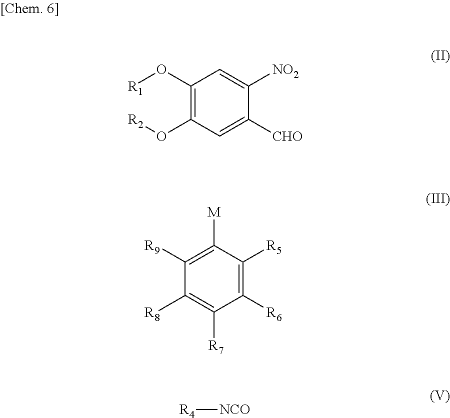

In the general formula (II), R₁ and R₂ are each the same as R₁ and R₂ in the general formula (I);

in the general formula (III), R₅ to R₉ are each the same as R₅ to R₉ in the general formula (I), M is a substituent group containing a metal and the metal is Mg, Zn, Li, Sn or Cu; and

in the general formula (V), R₄ is the same as R₄ in the general formula (I).

[8] A process for producing the N-(α-aromatic group-substituted-2-nitro-4,5-dialkoxybenzyloxycarbonyl)amine compound as described in the above [1], comprising reacting a carbinol compound represented by the following general formula (VI) with a compound represented by the following general formula (IV).

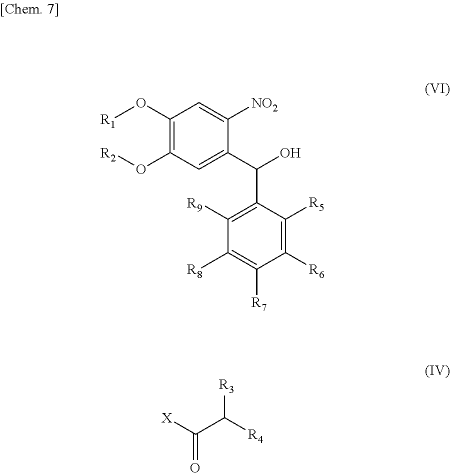

In the general formula (VI), R₁ and R₂ are each the same as R₁ and R₂ in the general formula (I) and R₅ to R₉ are each the same as R₅ to R₉ in the general formula (I); and

in the general formula (IV), R₃ and R₄ are each the same as R₃ and R₄ in the general formula (I), and X is a halogen atom selected from a fluorine atom, a chlorine atom, a bromine atom and an iodine atom.

[9] A process for producing the N-α-aromatic group-substituted-2-nitro-4,5-dialkoxybenzyloxycarbonyl) amine compound as described in the above [2], comprising the step of reacting a carbinol compound represented by the following general formula (VI) with an isocyanate compound represented by the following general formula (V).

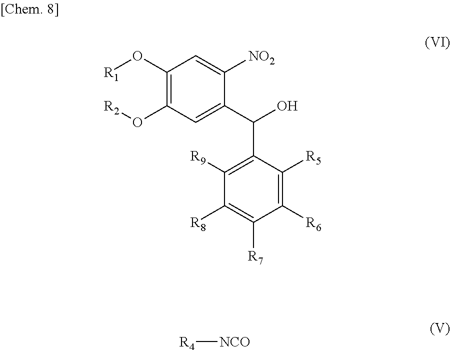

In the general formula (VI), R₁ and R₂ are each the same as R₁ and R₂ in the general formula (I), and R₅ to R₉ are each the same as R₅ to R₉ in the general formula (I); and

in the general formula (V), R₄ is the same as R₄ in the general formula (I).

[10] A process for producing the N-(α-aromatic group-substituted-2-nitro-4,5-dialkoxybenzyloxycarbonyl) amine compound as described in the above [1], comprising the steps of reacting a carbinol compound represented by the following general formula (VI) with a carbonyl compound represented by the following general formula (VII) to synthesize an ester compound represented by the following general formula (VIII) and

reacting the ester compound with an amine compound represented by the following general formula (IX).

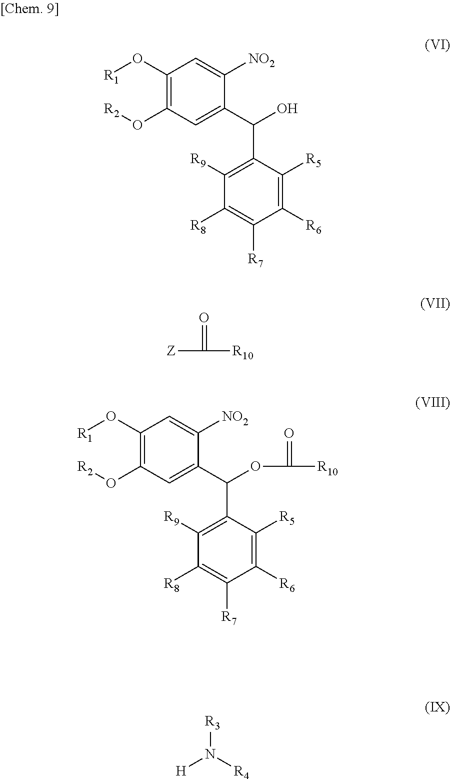

In the general formulae (VI) and (VIII), R₁ and R₂ are each the same as R₁ and R₂ in the general formula (I), and R₅ to R₉ are each the same as R₅ to R₉ in the general formula (I);

in the general formula (VII), Z is a chlorine atom, a bromine atom, an iodine atom, a trichloromethoxy group or a 1-imidazolyl group;

in the general formulae (VII) and (VIII), R₁₀ is a chlorine atom, trichloromethoxy group, 1-imidazolyl group, phenoxy group, 4-nitrophenoxy group or 4-cyanophenoxy group; and

in the general formula (IX), R₃ and R₄ are each the same as R₃ and R₄ in the general formula (I).

[11] The process for producing the N-(α-aromatic group-substituted-2-nitro-4,5-dialkoxybenzyloxycarbonyl)amine compound as described in the above [10], wherein the compound represented by the general formula (VII) is phosgene, trichloromethyl chloroformate, triphosgene, carbonyl diimidazole, p-nitrophenyl chloroformate or p-cyanophenyl chloroformate.

### EFFECT OF THE INVENTION

The N-(α-aromatic group-substituted-2-nitro-4,5-dialkoxybenzyloxycarbonyl)amine compound of the present invention is useful as a photobase generator, in particular useful as a photobase generator having sensitivity to h-ray. The N-(α-aromatic group-substituted-2-nitro-4,5-dialkoxybenzyloxycarbonyl)amine compound of the present invention can be employed, for example, as a component that constitutes a pattern formation material for use in photoresist or the like.

### BRIEF DESCRIPTION OF DRAWINGS

FIG. 1 is a ¹H-NMR spectrum of N-(α-phenyl-2-nitro-4,5-dimethoxybenzyloxycarbonyl)cyclohexyl amine obtained in Example 1.

FIG. 2 is a ¹H-NMR spectrum of N-(α-phenyl-2-nitro-4,5-dimethoxybenzyloxycarbonyl)morpholine obtained in Example 2.

FIG. 3 shows UV spectra of N-(α-phenyl-2-nitro-4,5-dimethoxybenzyloxycarbonyl)cyclohexyl amine obtained in Example 1, α-phenyl-2-nitro-4,5-dimethoxybenzyloxycarbonylmorpholine obtained in Example 2, and 2-nitro-4,5-dimethoxybenzyloxycarbonylcyclohexylamine obtained in Comparative Example 1.

FIG. 4 shows TG measurement results of N-(α-phenyl-2-nitro-4,5-dimethoxybenzyloxycarbonyl)cyclohexyl amine obtained in Example 1 and N-(α-phenyl-2-nitro-4,5-dimethoxybenzyloxycarbonyl)morpholine obtained in Example 2.

FIG. 5 is a ¹H-NMR spectrum of N-(α-phenyl-2-nitro-4,5-dimethoxybenzyloxycarbonyl)cyclohexyl methylamine obtained in Example 4.

FIG. 6 shows transmittance curves of Filter 1 and Filter 2 that were used in Examples 9 to 15 and Comparative Example 2.

### EMBODIMENT OF THE INVENTION

The present invention is described in detail below.

### N-(α-aromatic group-substituted-2-nitro-4,5-dialkoxybenzyloxycarbonyl)amine compound

The N-(α-aromatic group-substituted-2-nitro-4,5-dialkoxybenzyloxycarbonyl)amine compound of the present invention (hereinafter, also referred to simply as “the compound of the present invention”) is represented by the following general formula (I).

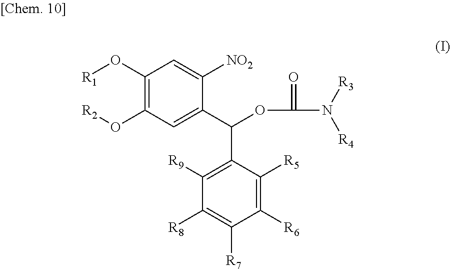

Alkoxy groups of OR₁ and OR₂ are introduced into a nitrobenzyl group, because of which the compound of the present invention absorbs light of a longer wavelength, as will be described later. Further, because an aromatic group is introduced at α-position of the nitrobenzyl group, the compound of the present invention has increased sensitivity to h-ray.

The compound of the present invention, through being irradiated with ultraviolet such as i-ray and h-ray, generates a conventionally known base, HNR₃R₄. This base functions as a catalyst for crosslinking reaction and polymerization reaction or as a crosslinking agent itself. Hereinafter, R₁ to R₉ in the above general formula (I) are described.

<As for R₁ and R₂>

In the above general formula (I), R₁ and R₂ are each independently an alkyl group having 1 to 12 carbon atoms which may have a substituent group or an aryl group having 6 to 12 carbon atoms which may have a substituent group, and R₁ and R₂ may be bonded to form an alkylene group having 1 to 12 carbon atoms which may have a substituent group or an arylene group having 6 to 12 carbon atoms which may have a substituent group. OR¹ and OR₂ constitute alkoxy groups. Because these alkoxy groups are introduced into a nitrobenzyl group, the compound represented by the general formula (I) absorbs light of a longer wavelength. Consequently, the compound of the present invention can absorb h-ray and generate a base.

Among the alkyl groups having 1 to 12 carbon atoms which may have a substituent group, an alkyl group having 1 to 6 carbon atoms which may have a substituent group is preferable and an alkyl group having 1 to 3 carbon atoms which may have a substituent group is more preferable, in view of the amount of a base generated per unit weight and easiness of the production. Examples of the substituent groups include methoxy group, phenyl group and 2-thioxanthyl group.

Examples of the alkyl groups having 1 to 12 carbon atoms which may have a substituent group include methyl group, ethyl group, n-propyl group, i-propyl group, n-butyl group and i-butyl group. Among them, the methyl group and ethyl group are preferable in view of the amount of a base generated per weight. The number of carbons of “the alkyl group having 1 to 12 (or 1 to 6, or 1 to 3) carbon atoms which may have a substituent group” is the number of carbons in the alkyl group part and does not include the number of carbons in the substituent group.

Among the aryl groups having 6 to 12 carbon atoms which may have a substituent group, an aryl group having 6 carbon atoms which may have a substituent group is preferable in view of the amount of a base generated per weight and easiness of the production. Examples of the substituent groups are those described in the explanation about the alkyl group having 1 to 12 carbon atoms.

Examples of the aryl groups having 6 to 12 carbon atoms which may have a substituent group include phenyl group, naphthyl group and toluoyl group. The number of carbons of “the aryl group having 6 to 12 (or 6) carbon atoms which may have a substituent group” is the number of carbons of the aryl group part and does not include the number of carbons in the substituent group.

As described above, R₁ and R₂ may be bonded to form an alkylene group having 1 to 12 carbon atoms which may have a substituent group or an arylene group having 6 to 12 carbon atoms which may have a substituent group. Examples of the substituent groups include methyl group, ethyl group, methoxy group and phenyl group. Examples of the alkylene groups or arylene groups formed when R₁ and R₂ are bonded include methylene group, ethylene group, 1,3-propylene group and 1,2-phenylene group. The number of carbons of “the alkylene group having 1 to 12 carbon atoms which may have a substituent group” and that of “the arylene group having 6 to 12 carbon atoms which may have a substituent group” are the number of carbons of the alkylene group part and the number of carbons of the arylene group part, respectively, and each do not include the number of carbons in the substituent groups.

<As for R₃ and R₄>

In the general formula (I), R₃ and R₄ are each independently a hydrogen atom, an alkyl group having 1 to 12 carbon atoms which may have a substituent group or an aryl group having 6 to 12 carbon atoms which may have a substituent group, at least one of R₃ and R₄ is not a hydrogen atom, and R₃ and R₄ may be bonded to form a cyclic structure which may contain a hetero atom.

Among the alkyl groups having 1 to 12 carbon atoms which may have a substituent group, an alkyl group having 1 to 8 carbon atoms which may have a substituent group is preferable and an alkyl group having 1 to 6 carbon atoms which may have a substituent group is more preferable, in view of the amount of a base generated per weight and easiness of the production. Examples of the substituent groups are those described in the explanation about the alkyl group having 1 to 12 carbon atoms which may have a substituent group with respect to R₁ and R₂.

Examples of the alkyl groups having 1 to 12 carbon atoms which may have a substituent group include cyclohexyl group in addition to the examples mentioned for the above R₁ and R₂. Preferable examples of the alkyl groups having 1 to 12 carbon atoms which may have a substituent group include cyclohexyl group, methyl group and ethyl group. The number of carbons of “the alkyl group having 1 to 12 (or 1 to 8, or 1 to 6) carbon atoms which may have a substituent group” is the number of carbons of the alkyl group part and does not include the number of carbons in the substituent group.

Among the aryl groups having 6 to 12 carbon atoms which may have a substituent group, an aryl group having 6 carbon atoms which may have a substituent group is preferable in view of the amount of a base generated per weight and easiness of the production. Examples of the substituent groups are those described in the explanation about the alkyl group having 1 to 12 carbon atoms with respect to R₁ and R₂.

Examples of the aryl groups having 6 to 12 carbon atoms which may have a substituent group include the examples mentioned above for R₁ and R₂. The number of carbons of “the aryl group having 6 to 12 (or 6) carbon atoms which may have a substituent group” is the number of carbons of the aryl group part and does not include the number of carbons in the substituent group.

As stated above, at least one of R₃ and R₄ is not a hydrogen atom. When both are hydrogen atoms, the compound of the general formula (I) has deteriorated stability and an amine generated through irradiation with ultraviolet is ammonia. This is why the compound of the general formula (I) wherein both R₃ and R₄ are hydrogen atoms is not useful as a base generator.

R₃ and R₄ may be bonded to form a cyclic structure which may contain a hetero atom, and a substituent may be bonded onto the ring. Examples of the substituent groups include the examples mentioned for the alkylene group having 1 to 12 carbon atoms which may have a substituent group or the arylene group having 6 to 12 carbon atoms which may have a substituent group formed b the bonding of R₁ and R₂, described in connection with R₁ and R₂. Examples of the groups constituted by R₃ and R₄ when R₃ and R₄ are bonded to form a cyclic structure include ethylene group, trimethylene group, tetramethylene group, pentamethylene group, 3-oxapentamethylene group and 1,5-dimethylpentamethylene group.

<As for R₅ to R₉>

In the general formula (I), R₅ to R₉ are each independently a hydrogen atom, an alkyl group having 1 to 12 carbon atoms, an aryl group having 6 to 12 carbon atoms, an alkoxy group having 1 to 12 carbon atoms, a halogen atom, a cyano group, an amino group, an alkylamino group having 1 to 12 carbon atoms, an acyloxy group having 1 to 12 carbon atoms, a nitro group or an acyl group having 1 to 12 carbon atoms.

Among the alkyl groups having 1 to 12 carbon atoms, an alkyl group having 1 to 6 carbon atoms is preferable and an alkyl group having 1 to 3 carbon atoms is more preferable, in view of the amount of a base generated per weight and easiness of the production. Examples of the alkyl groups having 1 to 12 carbon atoms include the examples mentioned for R₃ and R₄.

Among the aryl groups having 6 to 12 carbon atoms, an aryl group having 6 carbon atoms is preferable in view of the amount of a base generated per weight and easiness of the production. Examples of the aryl groups having 6 to 12 carbon atoms include phenyl group and naphthyl group.

Examples of the alkoxy groups having 1 to 12 carbon atoms include methoxy group and ethoxy group.

Examples of the halogen atoms include a chlorine atom and a bromine atom.

Examples of the alkyls of the alkylamino groups having 1 to 12 carbon atoms include methyl group, ethyl group and propyl group.

Examples of the acyloxy groups having 1 to 12 carbon atoms include acetoxyl group.

Examples of the acyl groups having 1 to 12 carbon atoms include formyl group, acetyl group and benzoyl group.

Because in the compound of the present invention, an aromatic group having the above-described R₅ to R₉ is introduced at α-position of the nitrobenzyl group, the compound has increased sensitivity to h-ray as is clear from the later-mentioned Examples.

### N-(α-aromatic group-substituted-2-nitro-4,5-dialkoxybenzyloxycarbonyl)amine compound

Specific examples of the compound of the present invention are as follows.

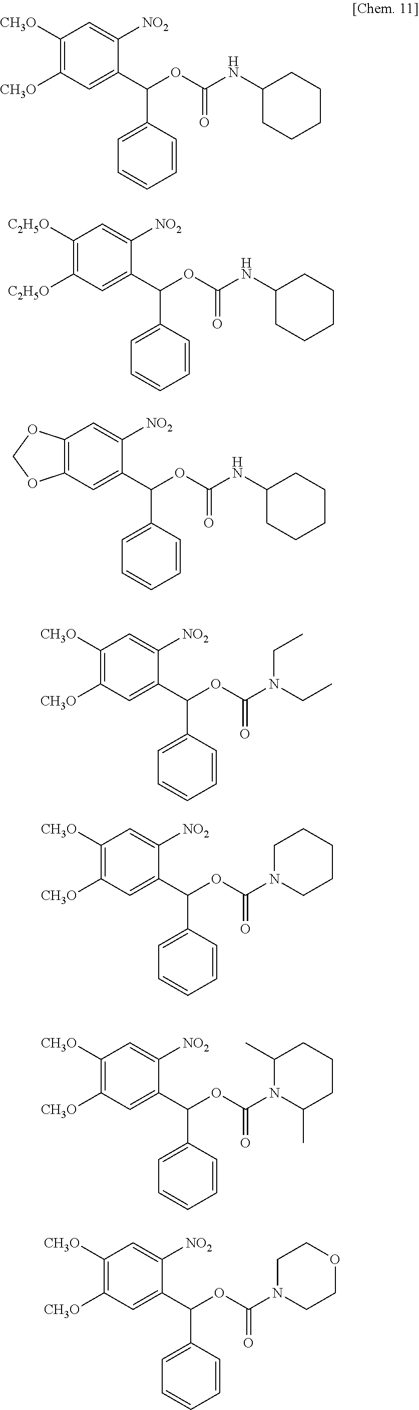

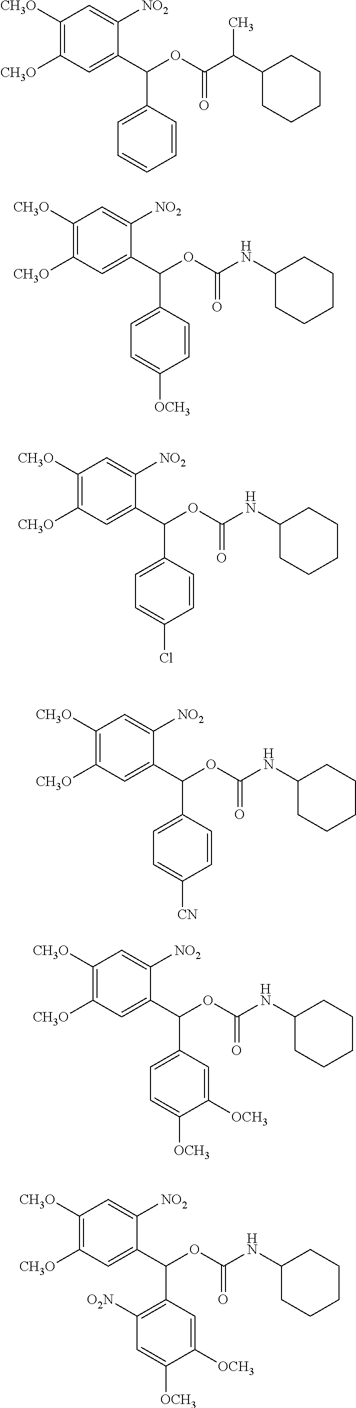

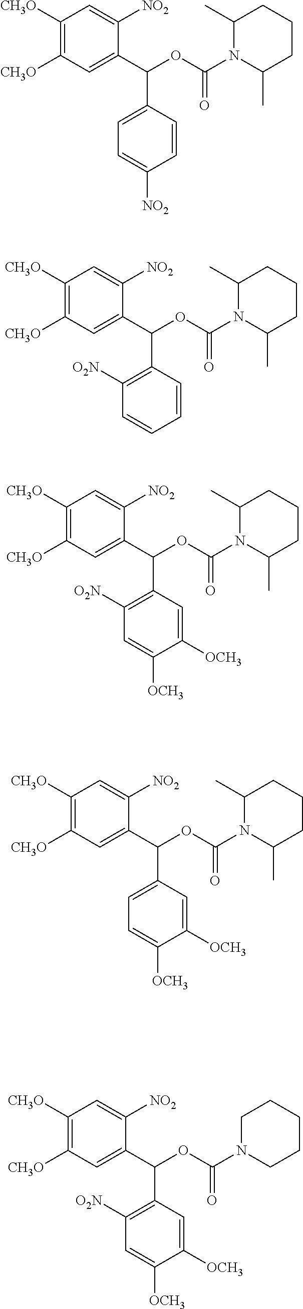

As described above, in the compound of the present invention, alkoxy groups of OR₂ and OR₂ are introduced into the nitrobenzyl group and an aromatic group is introduced at α-position of the nitrobenzyl group. Because of that, the compound has high sensitivity to not only i-ray but also h-ray. The compound generates a base (HNR₃R₄) upon irradiation with ultraviolet.

Further, the compound of the present invention has a high thermal resistance; specifically, the 5% weight loss temperature as measured by TG is usually 150° C. or higher and 300° C. or lower. The compound is, therefore, useful as a photopolymerization initiator of a composition which is supposed to, prior to polymerization, be heated in drying of a solvent after coating.

Particularly preferable examples of the compounds of the present invention as described above include
N-(α-phenyl-2-nitro-4,5-dimethoxybenzyloxycarbonyl)-cyclohexylamine,
N-(α-phenyl-2-nitro-4,5-dimethoxybenzyloxycarbonyl)-morpholine,
N-(α-phenyl-2-nitro-4,5-dimethoxybenzyloxycarbonyl)-cyclohexylmethylamine,
N-(α-phenyl-2-nitro-4,5-dimethoxybenzyloxycarbonyl)-2,6-dimethylpiperidine,
N-(α-(4-nitrophenyl)-2-nitro-4,5-dimethoxybenzyloxycarbonyl)-2,6-dimethylpiperidine,
N-(α-(2-nitrophenyl)-2-nitro-4,5-dimethoxybenzyloxycarbonyl)-2,6-dimethylpiperidine,
N-(α-(2-nitro-4,5-dimethoxyphenyl)-2-nitro-4,5-dimethoxybenzyloxycarbonyl)-2,6-dimethylpiperidine,
N-(α-phenyl-2-nitro-4,5-dimethoxybenzyloxycarbonyl)-piperidine, and
N-(α-(2-nitro-4,5-dimethoxyphenyl)-2-nitro-4,5-dimethoxybenzyloxycarbonyl)-piperidine.

### Process for producing N-(α-aromatic group-substituted-2-nitro-4,5-dialkoxybenzyloxycarbonyl)amine compound

### <Production Process 1>

The compound of the present invention can be produced by a method using a carbon nucleophilic agent such as a Grignard reactant. To be specific, an aldehyde compound represented by the following general formula (II) is reacted with an aromatic compound represented by the following general formula (III) and then the obtained product is reacted with a compound represented by the following general formula (IV) thereby to produce the compound of the present invention. The reaction product obtained by reacting the aldehyde compound represented by the general formula (II) with the aromatic compound represented by the general formula (III) may be isolated and reacted with the compound represented by the general formula (IV). The reaction may be carried out without the isolation.

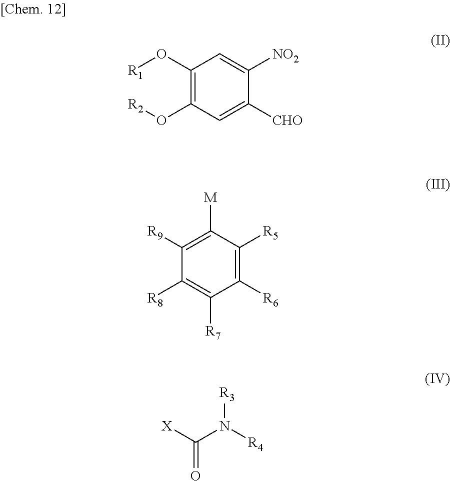

In the general formula (II), R₁ and R₂ are each the same as R₁ and R₂ in the general formula (I).

In the general formula (III), R₅ to R₉ are each the same as R₅ to R₉ in the general formula (I), M is a substituent group containing a metal, and the metal is Mg, Zn, Li, Sn or Cu. Examples of M include those in which a halogen atom or an alkoxy group is coordinated with the metals excluding Li; specific examples of M include Li, MgCl, MgBr and ZnCl.

In the general formula (IV), R₃ and R₄ are each the same as R₃ and R₄ in the general formula (I), and X is a halogen atom selected from a fluorine atom, a chlorine atom, a bromine atom and an iodine atom.

As a specific example of this reaction, 2-nitro-4,5-dimethoxybenzaldehyde is reacted with phenylmagnesium bromide, and the product, without being isolated, is reacted with morpholinecarbonylchloride thereby to obtain N-α-phenyl-2-nitro-4,5-dimethoxybenzyloxycarbonyl)morpholine (See the following formula).

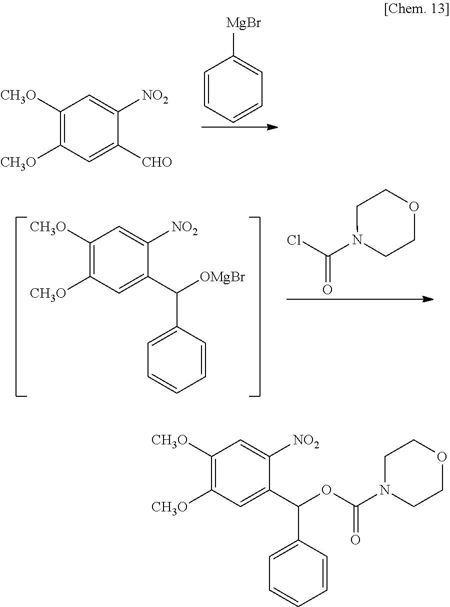

As another method, instead of the compound represented by the general formula (IV), an isocyanate compound represented by the following general formula (V) is reacted with a product obtained by reacting the aldehyde compound represented by the general formula (II) with the aromatic compound represented by the general formula (III). In this case, too, the reaction product obtained by reacting the aldehyde compound represented by the general formula (II) with the aromatic compound represented by the general formula (III) may be isolated and reacted with the compound represented by the general formula (V). This reaction can be carried out without the isolation.

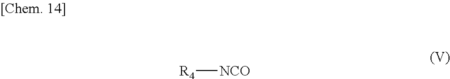

In the general formula (V), R₄ is the same as R₄ in the general formula (I). This reaction affords an N-(α-aromatic group-substituted-2-nitro-4,5-dialkoxybenzyloxycarbonyl)amine compound wherein in the general formula (I), R₃ is a hydrogen atom. A nitrogen atom, to which R₃ of the N-(α-aromatic group-substituted-2-nitro-4,5-dialkoxybenzyloxycarbonyl)amine compound wherein R₃ is a hydrogen atom thus obtained is bonded, has a nucleophilicity and therefore, can produce a nucleophilic substitution reaction with an alkane halide such as a methyl halide or an ethyl halide. As a result of the reaction, an N-(α-aromatic group-substituted-2-nitro-4,5-dialkoxybenzyloxycarbonyl)amine compound wherein R₃ is not a hydrogen atom is obtained. In this case, the nucleophilic substitution reaction can be more efficiently carried out by reacting the hydrogen atom of R₃ with lithium hydride or sodium hydride so as to replace it with lithium or sodium.

A specific example of a reaction where the aldehyde compound represented by the general formula (II) is reacted with the aromatic compound represented by the general formula (III) and subsequently the compound obtained by the reaction is reacted with the isocyanate compound represented by the general formula (V), is as follows. 2-nitro-4,5-dimethoxybenzaldehyde is reacted with phenyl magnesium bromide and the resultant product, with or without being isolated, is reacted with cyclohexylisocyanate thereby to obtain N-(α-phenyl-2-nitro-4,5-dimethoxybenzyloxycarbonyl)cyclohexyl amine.

As mentioned above, the Production Process 1 employs known reactions: a carbon nucleophilic reaction such as Grignard reaction and a nucleophilic addition of a hydroxyl group activated by a carbon nucleophilic agent. With the Production Process 1, the compound of the present invention can be produced through a simplified method which has only two stages if the isolation step is omitted.

The amount of the compound represented by the general formula (III) used in these reactions is not particularly limited, but is preferably 0.9 to 1.2 equivalents based on the aldehyde compound represented by the general formula (II). When the compound represented by the general formula (III) is used within such range, the product can be obtained at a good yield and a byproduct is generated in less amount.

The amount of the compound represented by the general formula (IV) or the general formula (V) used in these reactions is not particularly limited, but is preferably 1.0 to 1.2 equivalents based on the aldehyde compound represented by the general formula (II). When the compound represented by the general formula (IV) or the general formula (V) is used within such range, the product can be obtained at a good yield and a urea derivative which is a byproduct is hardly generated.

Any reaction solvent can be used in these reactions without limitation as long as the solvent can be used in a carbon nucleophilic reaction such as Grignard reaction. Specific examples thereof include diethylether, tetrahydrofuran and tetrahydropyran. However, the solvents that can be used are not limited to them.

The temperature in these reactions is not particularly limited, but is preferably 0° C. to 25° C. when the aldehyde compound represented by the general formula (II) is reacted with the aromatic compound represented by the general formula (III) and the product obtained is next, without being isolated, reacted with the compound represented by the general formula (IV) or the general formula (V). When the temperature is within this range, the reaction does not progress slowly and can afford the product at a good yield.

The reaction pressure in these reactions is not particularly limited, but is preferably normal pressure to 0.1 MPaG, more preferably normal pressure.

Regarding these reactions, the reaction time is usually 1 to 24 hours for the reaction of the aldehyde compound and the aromatic compound and usually 1 to 24 hours for the reaction of the compound obtained by the reaction and the compound represented by the general formula (IV) or (V).

When the isolation is carried out, preferable conditions under which the aldehyde compound represented by the general formula (II) is reacted with the aromatic compound represented by the general formula (III) are the same as those adopted when the isolation is not carried out; and preferable conditions under which the isolated product is reacted with the compound represented by the general formula (IV) or the general formula (V) are the same as preferable conditions in Production Process 2 that will be described below.

<Production Process 2>

In another embodiment, the compound of the present invention can be produced by reacting a carbinol compound represented by the following general formula (VI) with a compound represented by the following general formula (IV) or an isocyanate compound represented by the following general formula (V).

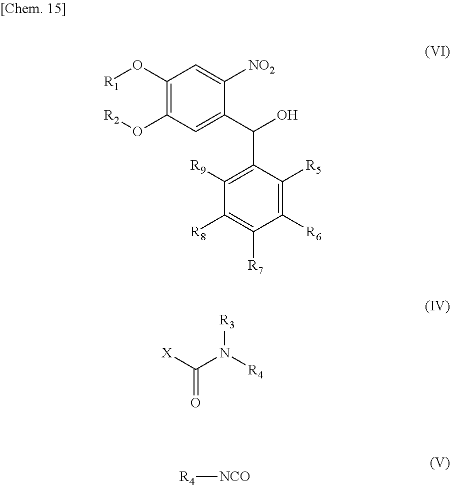

In the general formula (VI), R₁ and R₂ are each the same as R₁ and R₂ in the general formula (I), and R₅ to R₉ are each the same as R₅ to R₉ in the general formula (I). The compound represented by the general formula (VI) can be obtained by reacting the aldehyde compound represented by the general formula (II) and the aromatic compound represented by the general formula (III) as described in Production Process 1, and also can be synthesized by other known methods. For example, it can be synthesized by the methods described in Tetrahedron, 63, (2007), 474 and Molecules, 1999, 4, M113.

In the general formula (IV), R₃ and R₄ are each the same as R₃ and R₄ in the general formula (I), and X is a halogen atom selected from a fluorine atom, a chlorine atom, a bromine atom and an iodine atom.

In the general formula (V), R₄ is the same as R₄ in the general formula (I).

As mentioned above, in the Production Process 2, because the compound of the general formula (VI) is known, the compound of the present invention can be synthesized through a simplified method which has only one stage, by utilizing the well-known reaction, a nucleophilic addition of a hydroxyl group.

The used amount of the compound represented by the general formula (IV) or the isocyanate compound represented by the general formula (V) is not particularly limited, but is preferably 1.0 to 1.2 equivalents based on the carbinol compound represented by the general formula (VI). When the compound of the general formula (IV) or (V) is used within such range, the compound of the present invention can be obtained at a good yield and a urea derivative which is a byproduct is generated in less amount.

The reaction temperature in these reactions is not particularly limited, but is preferably 25° C. to 120° C. Within this temperature range, the reaction does not progress slowly and can afford the compound of the present invention at a good yield. The reaction pressure in these reactions is not particularly limited, but is preferably normal pressure to 0.1 MPaG, more preferably normal pressure. Further, the reaction time in these reactions is usually 1 to 24 hours.

The reaction using the compound represented by the general formula (IV) can accompany the addition of a basic compound for the purpose of facilitating the reaction through the neutralization of a hydrogen halide which is by-produced. The basic compound is not particularly limited but needs to be a compound which does not decompose the compound represented by the general formula (IV). The basic compound is preferably a tertiary amine compound, particularly preferably pyridine and triethylamine.

The reaction using the isocyanate compound represented by the general formula (V) may not necessarily accompany the use of a catalyst, but may accompany it to increase the reaction velocity. Examples of the catalysts include lithium chloride, lithium hydroxide and dibutyltin dilaurate. In the reaction using the isocyanate compound represented by the general formula (V), an N-(α-aromatic group-substituted-2-nitro-4,5-dialkoxybenzyloxycarbonyl)amine compound of the general formula (I) in which R₃ is a hydrogen atom is obtained. A nitrogen atom, to which R₃ of the N-α-aromatic group-substituted-2-nitro-4,5-dialkoxybenzyloxycarbonyl)amine compound wherein R₃ is a hydrogen atom thus obtained is bonded, has a nucleophilicity and therefore, can produce a nucleophilic substitution reaction with an alkane halide such as a methyl halide and an ethyl halide. As a result of the reaction, an N-(α-aromatic group-substituted-2-nitro-4,5-dialkoxybenzyloxycarbonyl)amine compound wherein R₃ is not a hydrogen atom is obtained. In this case, the nucleophilic substitution reaction can be more efficiently carried out by reacting the hydrogen atom of R₃ with lithium hydride or sodium hydride so as to replace it with lithium or sodium.

These reactions are usually carried out in a liquid phase, and as a reaction solvent, an aprotic solvent such as methylene chloride and toluene can be used.

<Production Process 3>

In a further embodiment, the compound of the present invention can be produced by reacting a carbinol compound represented by the following general formula (VI) with a carbonyl compound represented by the following general formula (VII) to synthesize an ester compound represented by the following general formula (VIII) and reacting the ester compound with an amine compound represented by the following general formula (IX). In this case, the ester compound obtained by reacting the carbinol compound with the carbonyl compound represented by the following general formula (VII) may be isolated and reacted with the amine compound represented by the general formula (IX). The reaction may be carried out without the isolation.

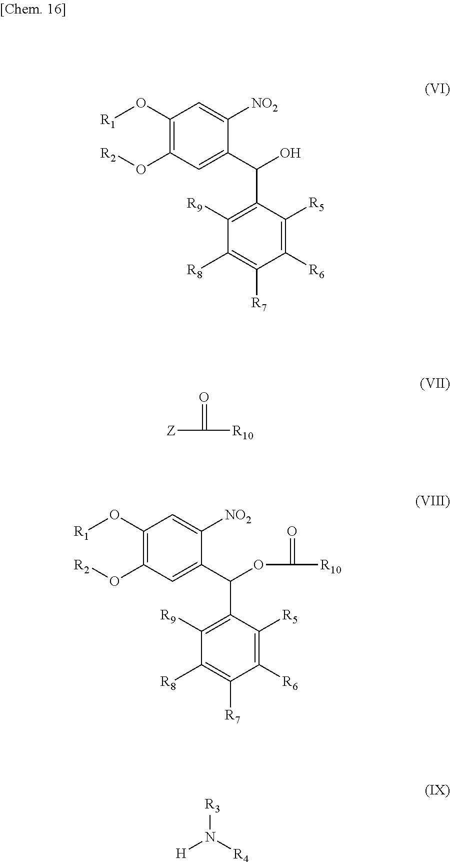

In the general formulae (VI) and (VIII), R₁ and R₂ are each the same as R₁ and R₂ in the general formula (I), and R₅ to R₉ are each the same as R₅ to R₉ in the general formula (I).

In the general formula (VII), Z is a chlorine atom, a bromine atom, an iodine atom, trichloromethoxy group or 1-imidazolyl group.

In the general formulae (VII) and (VIII), R₁₀ is a chlorine atom, trichloromethoxy group, 1-imidazolyl group, phenoxy group, 4-nitrophenoxy group or 4-cyanophenoxy group.

Examples of the compounds represented by the general formula (VII) are preferably phosgene, trichloromethyl chloroformate, triphosgene, carbonyl diimidazole, p-nitrophenyl chloroformate and p-cyanophenyl chloroformate. Because, in these example compounds, R₁₀ readily works as an elimination group in the reaction of the ester compound represented by the general formula (VIII) and the amine compound represented by the general formula (IX), and the example compounds are industrially available.

In the general formula (IX), R₃ and R₄ are each defined as R₃ and R₄ in the general formula (I).

As mentioned above, the Production Process 3 employs the carbonyl compound represented by the general formula (VII) so that the compound represented by the general formula (VI), a known compound, is converted into an ester compound having an N-(α-aromatic group-substituted-2-nitro-4,5-dialkoxybenzyloxycarbonyl) group in the compound of the present invention (the compound of the general formula (VIII)). The ester compound is reacted with the amine compound of the general formula (IX), in particular a secondary amine compound, whereby the corresponding secondary amine compound of the present invention can be synthesized. The Production Process 3 is particularly useful when it is difficult to obtain a carbamoyl chloride compound represented by the general formula (IV) in the Production Process 1 and Production Process 2.

The amount of the carbonyl compound used in the reaction of the carbinol compound represented by the general formula (VI) and the carbonyl compound represented by the general formula (VII) is not particularly limited, but is preferably 1.0 to 1.5 equivalents based on the carbinol compound represented by the general formula (VI). When the carbonyl compound is used within such range, the ester compound represented by the general formula (VIII) can be obtained at a good yield. In addition, it is easy to isolate the ester compound represented by the general formula (VIII) from the reaction mixture.

The amount of the amine compound used in the reaction of the ester compound represented by the general formula (VIII) and the amine compound represented by the general formula (IX) is not particularly limited, but is preferably 1.0 to 1.2 equivalents based on the ester compound. When the amine compound is used within such range, the compound of the present invention can be obtained at a good yield, and the compound of the present invention represented by the general formula (I) can be isolated with less amount of the contamination of the amine compound.

The reaction temperature in these reactions is not particularly limited, but is preferably −10° C. to 120° C., more preferably 0 to 80° C. When the temperature is within such range, the reaction does not progress slowly and can afford the compound of the present invention at a good yield.

The reaction pressure in these reactions is not particularly limited, but is preferably normal pressure to 0.1 MPaG, more preferably normal pressure.

Regarding the reaction time in these reactions, the reaction is usually 1 to 24 hours for the reaction of the carbinol compound and the carbonyl compound and usually 1 to 24 hours for the reaction of the carbonate ester compound obtained by the reaction and the amine compound.

The reaction using the carbonyl compound can accompany the addition of a basic compound for the purpose of facilitating the reaction. The basic compound is not particularly limited but needs to be a compound which does not decompose the carbonyl compound. The basic compound is preferably a tertiary amine compound, particularly preferably pyridine and triethylamine.

The reaction using the carbonyl compound may not necessarily accompany the use of a catalyst, but may accompany it to increase the reaction velocity. Examples of the catalysts include 4-(dimethylamino)pyridine and 2-(dimethylamino)pyridine.

The reaction of the ester compound and the amine compound may not necessarily accompany the use of a catalyst, but may accompany it to increase the reaction velocity. Examples of the catalysts include 1-hydroxybenzotriazole and 1-hydroxy-7-azabenzotriazole.

These reactions are usually carried out in a liquid phase, and as a reaction solvent, an aprotic solvent such as methylene chloride, N,N-dimethylacetamide and toluene can be used.

### Process for recovering an N-(α-aromatic group-substituted-2-nitro-4,5-dialkoxybenzyloxycarbonyl)amine compound

A process for recovering (purifying) the compound of the present invention obtained by the above-described Production Processes 1 to 3 is not particularly limited. The compound of the present invention can be recovered (purified) at a good purity through purification means such as column chromatography, extraction, recrystallization or reprecipitation.

### EXAMPLES

Hereinafter, the present invention is described in greater detail with reference to Examples. The present invention, however, is not limited to these Examples.

### Example 1

### Synthesis of N-(α-phenyl-2-nitro-4,5-dimethoxybenzyloxycarbonyl)cyclohexyl amine

10.9 g of 2-nitro-4,5-dimethoxybenzaldehyde (a reagent manufactured by Tokyo Chemical Industry Co., Ltd.) was dissolved in 150 mL of anhydrous tetrahydrofuran. The mixture was cooled down to 0° C. in nitrogen stream while stirring. Then, 50 mL of tetrahydrofuran solution of phenylmagnesium bromide (1 mol/L, manufactured by Aldrich) was dropped thereto over 15 minutes.

After the completion of the dropping, to the reaction solution, 7.51 g of cyclohexylisocyanate (a reagent manufactured by Tokyo Chemical Industry Co., Ltd.) was added at room temperature. Then, the mixture was stirred for 5 hours and allowed to stand overnight.

On the following day, an aqueous solution (which used 100 mL of water) in which 3.2 g of ammonium chloride was dissolved was added, and the mixture was stirred for 10 minutes and then subjected to extraction using ethyl acetate. The organic phase was washed with an aqueous solution of saturated sodium hydrogen carbonate, washed with water and concentrated with an evaporator, whereby a pale-yellow solid was obtained. The obtained solid was purified by column chromatography using a mixed solvent of hexane and ethyl acetate, and the fraction was concentrated to obtain 1.2 g of a light-yellow crystal.

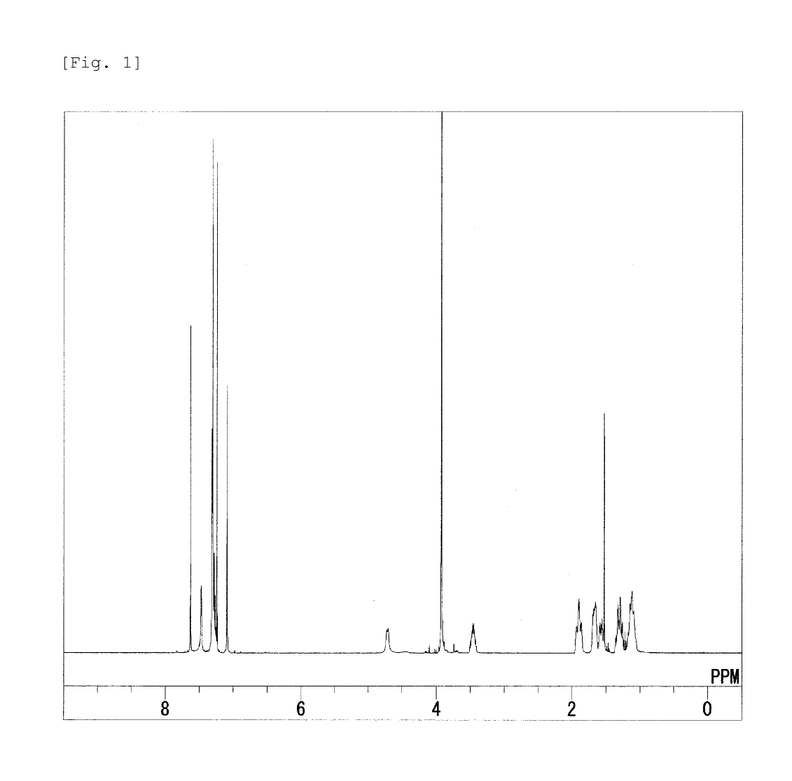

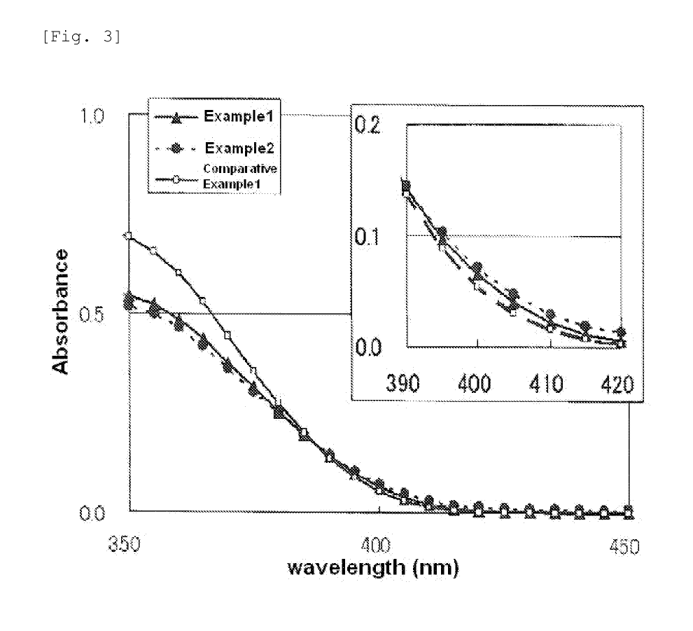

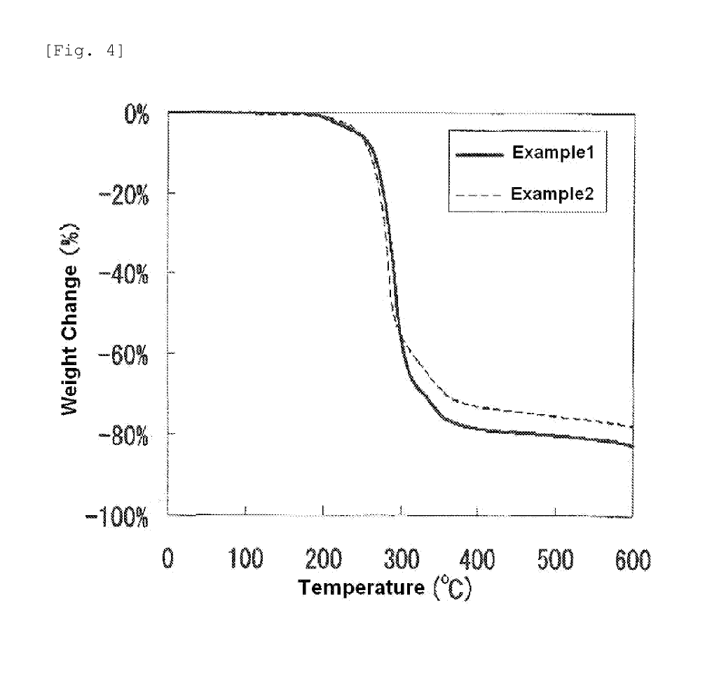

This crystal was confirmed by ¹H-NMR to be N-(α-phenyl-2-nitro-4,5-dimethoxybenzyloxycarbonyl)cyclohexyl amine. The ¹H-NMR spectrum of this compound is shown in FIG. 1 (1.0-2.0 ppm m 10H —CH₂—, 3.5 ppm m 1H —CH—N, 3.9 ppm s 6H OCH₃, 4.7 ppm d 1H NH, 7.1 ppm s 1H CH—O, 7.2-7.7 ppm 7H aromatic C—H). The HPLC purity was 95 area %. The isolation yield based on 2-nitro-4,5-dimethoxybenzaldehyde was 6%. The melting point of this compound was 176° C. The UV absorption spectrum of this compound is shown in FIG. 3 (1×10⁻⁴ mol/L acetonitrile solution). According to the UV spectrum, this compound was found to have absorption at 405 nm. Further, according to the TG measurement of this compound, the 5% weight loss temperature thereof was 209° C. (FIG. 4).

### Example 2

### Synthesis of N-(α-phenyl-2-nitro-4,5-dimethoxybenzyloxycarbonyl)morpholine

The same procedure as in Example 1 was repeated except that 7.5 g of morpholinecarbonylchloride was used instead of cyclohexylisocyanate, whereby 9.6 g of a light-yellow crystal was obtained.

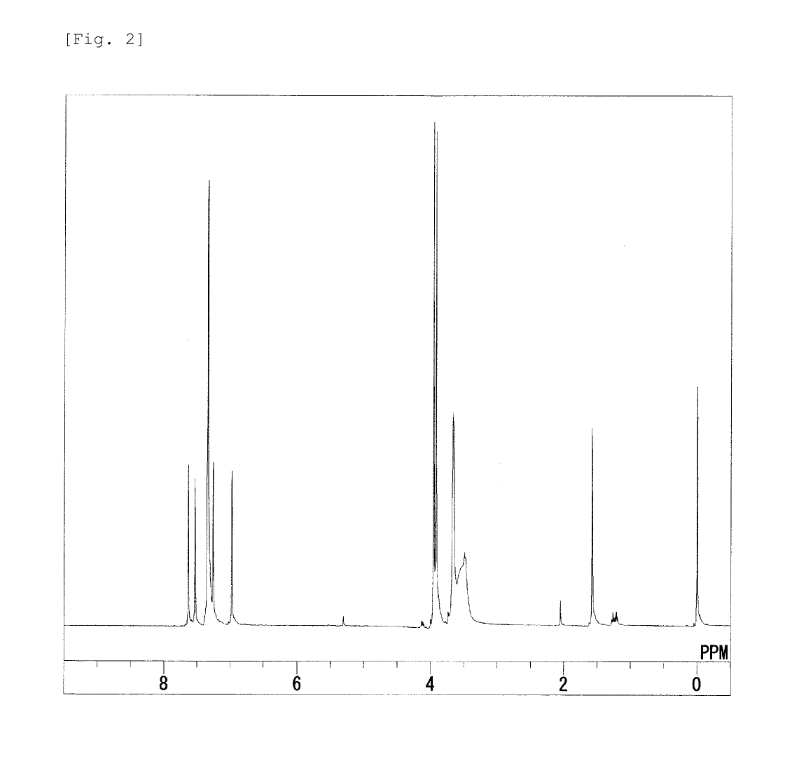

This crystal was confirmed by ¹H-NMR to be N-(α-phenyl-2-nitro-4,5-dimethoxybenzyloxycarbonyl)morpholine. The HPLC purity was 98 area %. The isolation yield based on 2-nitro-4,5-dimethoxybenzaldehyde was 47%. The ¹H-NMR spectrum of this compound is shown in FIG. 2 (3.4-3.7 ppm 8H OCH₂CH₂N, 3.9 ppm 6H OCH₃, 6.9 ppm s 1H CH—O, 7.2-7.7 ppm 7H aromatic CH). The UV absorption spectrum of this compound is shown in FIG. 3 (1×10⁻⁴ mol/L acetonitrile solution). According to the UV spectrum, this compound was found to have absorption at 405 nm. The result of the TG measurement of this compound is shown in FIG. 4. The 5% weight loss temperature of this compound was 233° C.

### Comparative Example 1

### Synthesis of N-(2-nitro-4,5-dimethoxybenzyloxycarbonyl)cyclohexylamine

With reference to JP-A-H06-345711, 9.53 g of N-(2-nitro-4,5-dimethoxybenzyloxycarbonyl)cyclohexylamine was obtained from 6.24 g of 2-nitro-4,5-dimethoxybenzylalcohol and 5.13 g of cyclohexylisocyanate. The UV absorption spectrum of this compound is shown in FIG. 3 (1×10⁻⁴ mol/L acetonitrile solution). It was found that this compound had an absorbance at 405 nm that was weaker than those of the compounds of Example 1 and Example 2.

### Example 3

### Synthesis of N-(α-phenyl-2-nitro-4,5-dimethoxybenzyloxycarbonyl)cyclohexyl amine

α-phenyl-2-nitro-4,5-dimethoxybenzylalcohol, a known compound, was synthesized with reference to Tetrahedron, 63, (2007), 474 and Molecules, 1999, 4, M113. 0.26 g of α-phenyl-2-nitro-4,5-dimethoxybenzylalcohol and 0.17 g of cyclohexylisocyanate were added to dehydrated toluene (30 mL) together with 0.06 g of dibutyltin dilaurate. The mixture was heated and reacted under reflux for 10 hours.

The obtained reaction liquid was concentrated. Then, the obtained solid was dissolved in methylene chloride (30 mL) and washed with saturated saline water (20 mL) and water (20 mL). The methylene chloride layer was concentrated and recrystallized with ethanol, whereby 0.19 g of a light-yellow crystal was obtained.

This crystal was confirmed by ¹H-NMR to be N-(α-phenyl-2-nitro-4,5-dimethoxybenzyloxycarbonyl)cyclohexyl amine. The HPLC purity was 98.5 area %. The isolation yield based on α-phenyl-2-nitro-4,5-dimethoxybenzylalcohol was 50%.

### Example 4

### Synthesis of N-(α-phenyl-2-nitro-4,5-dimethoxybenzyloxycarbonyl)cyclohexyl methylamine

Into a 100 mL two-neck flask, 1.25 g of N-α-phenyl-2-nitro-4,5-dimethoxybenzyloxycarbonyl)cyclohexyl amine synthesized in Example 1, tetrahydrofuran (19.8 mL) and dimethylformamide (2.0 mL) were added and dissolved. While cooling with ice, 0.144 g of sodium hydride was introduced thereto. Thereafter, methyl iodide (0.56 mL) was introduced thereto. The mixture was stirred at 0° C. for 10 minutes, and heated under reflux for 7 hours.

The mixture was allowed to cool and then a solid was precipitated. Hence, it was dissolved by adding 10 mL of dimethylformamide. The reaction liquid was added into 10 wt % hydrochloric acid aqueous solution (22 mL). The mixture was subjected to extraction by adding ethyl acetate (22 mL). The organic layer was dehydrated over anhydrous magnesium sulfate. The obtained solid was purified by silica gel chromatography, and the fraction was concentrated, whereby 0.76 g of a pale-yellow solid was obtained.

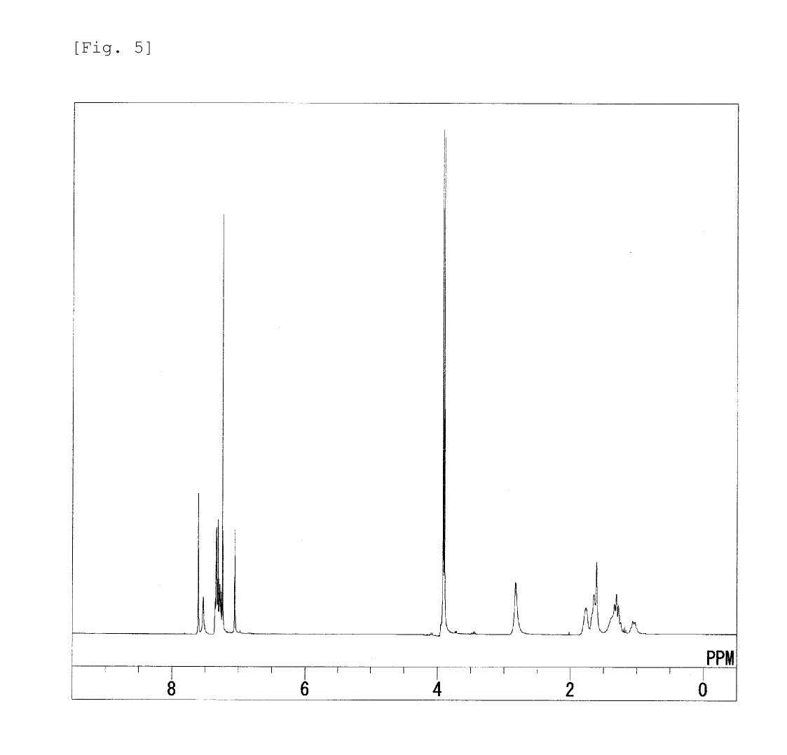

The obtained solid was identified by ¹H-NMR to be N-(α-phenyl-2-nitro-4,5-dimethoxybenzyloxycarbonyl)cyclohexyl methylamine. The ¹H-NMR spectrum of this compound is shown in FIG. 5. The isolation yield based on N-(α-phenyl-2-nitro-4,5-dimethoxybenzyloxycarbonyl)cyclohexyl amine was 59%.

### Example 5

### Synthesis of N-(α-phenyl-2-nitro-4,5-dimethoxybenzyloxycarbonyl)-2,6-dimethylpiperidine

Into a 200 mL two-neck flask, 5.8 g of α-phenyl-2-nitro-4,5-dimethoxybenzylalcohol, 4.4 g of p-nitrophenyl chloroformate and 0.1 g of N,N-dimethyl-4-aminopyridine (DMAP) were added. Then, while cooling with ice, into the mixture, a mixed liquid of 80 mL of anhydrous N,N-dimethylacetamide and 4.1 g of triethylamine was dropped under nitrogen stream, and the mixture was stirred for 3 hours. Then, after stirring at room temperature for 2 hours, 1.4 g of nitrophenyl chloroformate was further added thereto, and the reaction solution was stirred overnight.

On the following day, the reaction solution was added into 1.5 L of iced water and stirred until the ice melted. Thereafter, the solution was subjected to suction filtration, and the obtained solid was washed with water. The solid was subjected to extraction using ethyl acetate. The organic layer was dehydrated over sodium sulfate, and the resultant was concentrated with an evaporator, whereby 11.2 g of a yellow solid was obtained.

The yellow solid was washed with a mixed solvent of hexane and ethyl acetate (volume ratio of 1:1), whereby α-phenyl-2-nitro-4,5-dimethoxybenzyl-4-nitrophenylcarbonate was obtained as a light-yellow green solid. The HPLC purity was 97.7 area % and the isolation yield was 50%.

4.5 g of α-phenyl-2-nitro-4,5-dimethoxybenzyl-4-nitrophenylcarbonate, 0.4 g of 1-hydroxy-7-azabenzotriazole (HOAt), 6.7 g of cis-2,6-dimethylpiperidine and 50 mL of anhydrous N,N-dimethylacetamide were added into a 300 mL flask and stirred under nitrogen stream at 60° C. for 3 hours, and then stirred at 70° C. for 1 hour.

The reaction solution was added into 1.4 L of 1 wt % sodium hydrogen carbonate. The precipitated solid was subjected to suction filtration. The solid was washed with 1 wt % sodium hydrogen carbonate until the filtrate became colorless and transparent, and washed with water.

The obtained solid was transferred to a conical flask, into which 200 mL of ethyl acetate was added. The mixture was dehydrated over sodium sulfate and concentrated with an evaporator. The obtained solid was purified with a moderate pressure preparative chromatography (manufactured by Yamazen Corporation, YFLC-Eprep), and the fraction was concentrated, whereby 3.6 g of a solid having HPLC purity of 97.2 area % was obtained.

Further, it was recrystallized with a mixed solvent of ethanol and hexane (volume ratio of 1:8) to obtain 3.1 g of N-(α-phenyl-2-nitro-4,5-dimethoxybenzyloxycarbonyl)-2,6-dimethylpiperidine as a light-yellow crystal. The HPLC purity was 98.5 area % and the isolation yield based on α-phenyl-2-nitro-4,5-dimethoxybenzylalcohol was 36%. This compound was identified by ¹H-NMR (1.0 ppm d 3H —CH₃, 1.3 ppm d 3H —CH₃, 1.4-1.9 ppm m 6H —CH₂—, 3.9 ppm s 6H OCH₃, 3.9 ppm s 6H OCH₃, 4.4 ppm m 2H —CH—N, 7.1 ppm s 1H CH—O, 7.2-7.7 ppm 7H aromatic C—H).

### Example 6

### Synthesis of N-(α-(4-nitrophenyl)-2-nitro-4,5-dimethoxybenzyloxycarbonyl)-2,6-dimethylpiperidine

The same procedure as in Example 5 was repeated except that α-(4-nitrophenyl)-2-nitro-4,5-dimethoxybenzylalcohol was used instead of α-phenyl-2-nitro-4,5-dimethoxybenzylalcohol, whereby N-(α-(4-nitrophenyl)-2-nitro-4,5-dimethoxybenzyloxycarbonyl)-2,6-dimethylpiperidine was synthesized (isolation yield: 33%). This compound was identified by ¹H-NMR (1.1 ppm d 3H —CH₃, 1.3 ppm d 3H —CH₃, 1.4-1.9 ppm m 6H —CH₂—, 3.9 ppm s 3H OCH₃, 3.9 ppm s 3H OCH₃, 4.4 ppm m 2H —CH—N, 7.1 ppm s 1H CH—O, 7.5-8.2 ppm 6H aromatic C—H).

### Example 7

### Synthesis of N-(α-(2-nitro-4,5-dimethoxyphenyl)-2-nitro-4,5-dimethoxybenzyloxycarbonyl)-2,6-dimethylpiperidine

The same procedure as in Example 5 was repeated except that α-(2-nitro-4,5-dimethoxyphenyl)-2-nitro-4,5-dimethoxybenzylalcohol was used instead of α-phenyl-2-nitro-4,5-dimethoxybenzylalcohol, whereby N-(α-(2-nitro-4,5-dimethoxyphenyl)-2-nitro-4,5-dimethoxybenzyloxycarbonyl)-2,6-dimethylpiperidine was synthesized (isolation yield: 16%). This compound was identified by ¹H-NMR (1.3 ppm d 6H —CH₃, 1.4-1.9 ppm m 6H —CH₂—, 3.7 ppm s 6H OCH₃, 4.0 ppm s 6H OCH₃, 4.3 ppm m 2H —CH—N, 6.7 ppm s 2H aromatic C—H, 7.7 ppm s 2H aromatic C—H, 7.9 ppm s 1H CH-0).

### Example 8

### Synthesis of N-(α-phenyl-2-nitro-4,5-dimethoxybenzyloxycarbonyl)piperidine

The same procedure as in Example 5 was repeated except that piperidine was used instead of cis-2,6-dimethylpiperidine, whereby N-(α-phenyl-2-nitro-4,5-dimethoxybenzyloxycarbonyl)piperidine was synthesized (isolation yield: 16%). This compound was identified by ¹H-NMR (1.4-1.8 ppm m 6H —CH2-, 3.5 ppm br 4H —CH2-N, 3.9 ppm s 3H OCH₃, 3.9 ppm s 3H OCH₃, 7.0 ppm s 1H CH—O, 7.2-7.7 ppm 7H aromatic C—H).

### Example 9

### Measurement of Photo-Degradative Ability

1.0 mg of the N-(α-phenyl-2-nitro-4,5-dimethoxybenzyloxycarbonyl)cyclohexylamine obtained in Example 1 was weighed using an electronic scale into a quartz-made NMR tube and was dissolved by adding 0.5 mL of heavy acetonitrile.

This sample was irradiated, through a filter 1 which did not transmit light of wavelength of not more than 350 nm, with light of entire wavelength of a high-pressure mercury vapor lamp (manufactured by Ushio Inc., SPOT CURE SP-III 250UA, lamp model number: USH-255BY) which was set such that light would have 100 J/cm² (in terms of i-ray; ultraviolet intensity meter: UIT-150 manufactured by Ushio Inc.; photoreceiver: UVD-5365) before transmitting through the filter and such that the light would have 18.2 J/cm² (in terms of i-ray; ultraviolet intensity meter: UIT-150 manufactured by Ushio Inc.; photoreceiver: UVD-5365) after transmitting through the filter. NMR spectra of the sample before irradiation with the light and the sample after irradiation with the light were compared, whereby the photo-degradative property of the N-(α-phenyl-2-nitro-4,5-dimethoxybenzyloxycarbonyl)cyclohexyl amine in the wavelength region of not less than i-ray (365 nm) was evaluated.

In a similar manner, a sample was irradiated, through a filter 2 which did not transmit light of wavelength of not more than 380 nm, with light of entire wavelength of the high-pressure mercury vapor lamp which was set such that the light would have 100 J/cm² (in terms of i-ray; ultraviolet intensity meter: UIT-150 manufactured by Ushio Inc.; photoreceiver: UVD-S365) and 470 J/cm² (in terms of h-ray; ultraviolet intensity meter: UIT-101 manufactured by Ushio Inc.; photoreceiver: UVD-405PD) before transmitting through the filter; and such that the light would have 0 J/cm² (in terms of i-ray; ultraviolet intensity meter: UIT-150 manufactured by Ushio Inc.; photoreceiver: UVD-S365) and 160 J/cm² (in terms of h-ray; ultraviolet intensity meter: UIT-101 manufactured by Ushio Inc.; photoreceiver: UVD-405PD) after transmitting through the filter. NMR spectra of the sample before irradiation with the light and the sample after irradiation with the light were compared, whereby the photo-degradative property of the N-(α-phenyl-2-nitro-4,5-dimethoxybenzyloxycarbonyl)cyclohexyl amine in the wavelength region of not less than h-ray (405 nm) was evaluated.

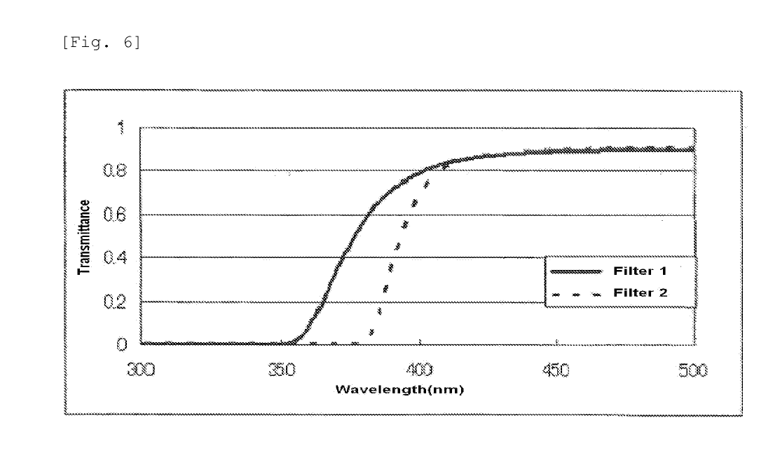

The transmittance curves of the filter 1 and the filter 2 are shown in FIG. 6. In addition, the results of the evaluation of the photo-degradative property are shown in the following Table 1.

### Examples 10 to 15

The same procedure as in Example 9 was repeated, except that the photobase generators indicated in the following Table 1 (N-(α-aromatic group-substituted-2-nitro-4,5-dialkoxybenzyloxycarbonyl)amine compounds) were used, to evaluate the photo-degradative properties. The results are shown in the following Table 1.

### Comparative Example 2

The same procedure as in Example 9 was repeated, except that the photobase generator synthesized in Comparative Example 1 (N-(2-nitro-4,5-dimethoxybenzyloxycarbonyl)cyclohexylamine) was used, to evaluate the photo-degradative property. The result is shown in the following Table 1.

TABLE 1

| | | Photo-degradation rate | |
|---|---|---|---|
| | Photobase generator | Filter 1 | Filter 2 |
| Example 9 | Compound of | 50% | 40% |
| | Example 1 | | |
| Example 10 | Compound of | 50% | 40% |
| | Example 2 | | |
| Example 11 | Compound of | 40% | 20% |
| | Example 4 | | |
| Example 12 | Compound of | 60% | 30% |
| | Example 5 | | |
| Example 13 | Compound of | 60% | 20% |
| | Example 6 | | |
| Example 14 | Compound of | 80% | 20% |
| | Example 7 | | |
| Example 15 | Compound of | 30% | 20% |
| | Example 8 | | |
| Comparative | Compound of | 10% | Not |
| Example 2 | Comparative | | degraded |
| | Example 1 | | |

It is clear from Table 1 that the N-(α-aromatic group-substituted-2-nitro-4,5-dialkoxybenzyloxycarbonyl)amine compound of the present invention is degraded also by being irradiated with the light that has transmitted through the filter 2 which does not transmit the light of a wavelength of not more than 380 nm including i-ray.

In view of this result and the results of UV absorption spectra shown in FIG. 4 in combination, it is considered that the N-(α-aromatic group-substituted-2-nitro-4,5-dialkoxybenzyloxycarbonyl)amine compound of the present invention is degraded by being irradiated with the light of a wavelength of 405 nm, i.e., h-ray, to generate a base.

1. An N-(α-aromatic group-substituted-2-nitro-4,5-dialkoxybenzyloxycarbonyl)amine compound represented by the following general formula (I): 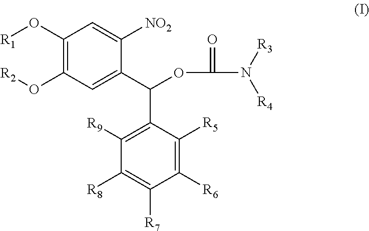
wherein in the general formula (I), R₁ and R₂ are each independently an alkyl group having 1 to 12 carbon atoms which may have a substituent group or an aryl group having 6 to 12 carbon atoms which may have a substituent group, and R₁ and R₂ may be bonded to form an alkylene group having 1 to 12 carbon atoms which may have a substituent group or an arylene group having 6 to 12 carbon atoms which may have a substituent group;
R₃ and R₄ are each independently a hydrogen atom, an alkyl group having 1 to 12 carbon atoms which may have a substituent group or an aryl group having 6 to 12 carbon atoms which may have a substituent group, at least one of R₃ and R₄ is not a hydrogen atom, and R₃ and R₄ may be bonded to form a cyclic structure which may contain a hetero atom; and
R₅ to R₉ are each independently a hydrogen atom, an alkyl group having 1 to 12 carbon atoms, an aryl group having 6 to 12 carbon atoms, an alkoxy group having 1 to 12 carbon atoms, a halogen atom, a cyano group, an amino group, an alkylamino group having 1 to 12 carbon atoms, an acyloxy group having 1 to 12 carbon atoms, a nitro group or an acyl group having 1 to 12 carbon atoms.

2. The N-(α-aromatic group-substituted-2-nitro-4,5-dialkoxybenzyloxycarbonyl)amine compound according to claim 1, wherein in the general formula (I), R₃ is a hydrogen atom.

3. The N-(α-aromatic group-substituted-2-nitro-4,5-dialkoxybenzyloxycarbonyl)amine compound according to claim 1, wherein in the general formula (I), R₁ and R₂ are methyl groups, R₃ and R₄ are bonded to form a morpholyl group and R₅ to R₉ are all hydrogen atoms.

4. The N-(α-aromatic group-substituted-2-nitro-4,5-dialkoxybenzyloxycarbonyl)amine compound according to claim 1, wherein in the general formula (I), R₁ and R₂ are methyl groups, R₃ is a hydrogen atom, R₄ is a cyclohexyl group and R₅ to R₉ are all hydrogen atoms.

5. The N-(α-aromatic group-substituted-2-nitro-4,5-dialkoxybenzyloxycarbonyl)amine compound according to claim 1, wherein the compound represented by the general formula (I) is at least one compound selected from the group consisting of
N-(α-phenyl-2-nitro-4,5-dimethoxybenzyloxycarbonyl)-2,6-dimethylpiperidine,
N-(α-(4-nitrophenyl)-2-nitro-4,5-dimethoxybenzyloxycarbonyl)-2,6-dimethylpiperidine,
N-(α-(2-nitrophenyl)-2-nitro-4,5-dimethoxybenzyloxycarbonyl)-2,6-dimethylpiperidine,
N-(α-(2-nitro-4,5-dimethoxyphenyl)-2-nitro-4,5-dimethoxybenzyloxycarbonyl)-2,6-dimethylpiperidine,
N-(α-phenyl-2-nitro-4,5-dimethoxybenzyloxycarbonyl)-piperidine and
N-(α-(2-nitro-4,5-dimethoxyphenyl)-2-nitro-4,5-dimethoxybenzyloxycarbonyl)-piperidine.

6. A process for producing the N-(α-aromatic group-substituted-2-nitro-4,5-dialkoxybenzyloxycarbonyl)amine compound according to claim 1, comprising reacting an aldehyde compound represented by the following general formula (II) and an aromatic compound represented by the following general formula (III) and reacting the compound obtained by the reaction with a compound represented by the following general formula (IV): 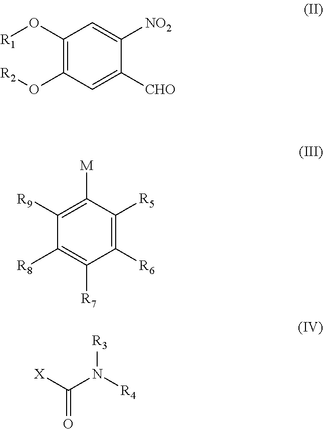
wherein in the general formula (II), R₁ and R₂ are each the same as R₁ and R₂ in the general formula (I);
in the general formula (III), R₅ to R₉ are each the same as R₅ to R₉ in the general formula (I), M is a substituent group containing a metal, and the metal is Mg, Zn, Li, Sn or Cu; and
in the general formula (IV), R₃ and R₄ are each the same as R₃ and R₄ in the general formula (I), and X is a halogen atom selected from a fluorine atom, a chlorine atom, a bromine atom and an iodine atom.

7. A process for producing the N-(α-aromatic group-substituted-2-nitro-4,5-dialkoxybenzyloxycarbonyl)amine compound according to claim 2, comprising reacting an aldehyde compound represented by the following general formula (II) with an aromatic compound represented by the following general formula (III) and reacting the compound obtained by the reaction with an isocyanate compound represented by the following general formula (V): 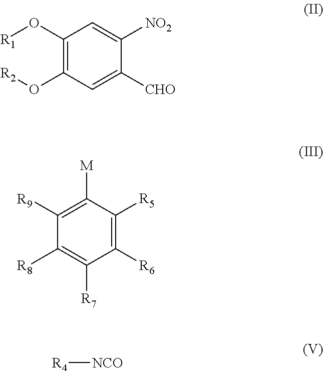
wherein in the general formula (II), R₁ and R₂ are each the same as R₁ and R₂ in the general formula (I);
in the general formula (III), R₅ to R₉ are each the same as R₅ to R₉ in the general formula (I), M is a substituent group containing a metal and the metal is Mg, Zn, Li Sn or Cu; and
in the general formula (V), R₄ is the same as R₄ in the general formula (I).

8. A process for producing the N-(α-aromatic group-substituted-2-nitro-4,5-dialkoxybenzyloxycarbonyl)amine compound according to claim 1, comprising reacting a carbinol compound represented by the following general formula (VI) with a compound represented by the following general formula (IV), 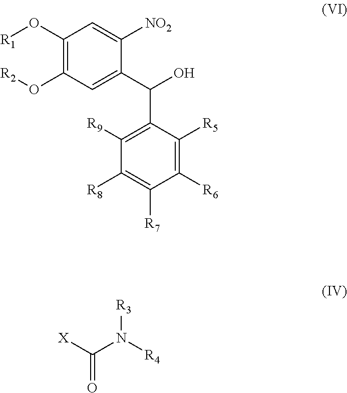
wherein in the general formula (VI), R₁ and R₂ are each the same as R₁ and R₂ in the general formula (I) and R₅ to R₉ are each the same as R₅ to R₉ in the general formula (I); and
in the general formula (IV), R₃ and R₄ are each the same as R₃ and R₄ in the general formula (I), and X is a halogen atom selected from a fluorine atom, a chlorine atom, a bromine atom and an iodine atom.

9. A process for producing the N-(α-aromatic group-substituted-2-nitro-4,5-dialkoxybenzyloxycarbonyl)amine compound according to claim 2, comprising reacting a carbinol compound represented by the following general formula (VI) with an isocyanate compound represented by the following general formula (V): 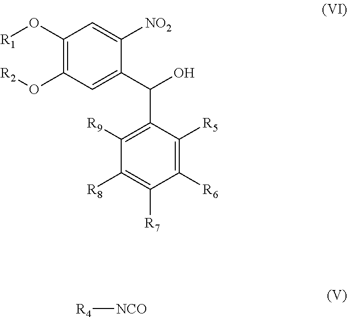
wherein in the general formula (VI), R₁ and R₂ are each the same as R₁ and R₂ in the general formula (I), and R₅ to R₉ are each the same as R₅ to R₉ in the general formula (I); and
in the general formula (V), R₄ is the same as R₄ in the general formula (I).

10. A process for producing the N-(α-aromatic group-substituted-2-nitro-4,5-dialkoxybenzyloxycarbonyl)amine compound according to claim 1, comprising reacting a carbinol compound represented by the following general formula (VI) with a carbonyl compound represented by the following general formula (VII) to synthesize an ester compound represented by the following general formula (VIII) and
reacting the ester compound with an amine compound represented by the following general formula (IX): 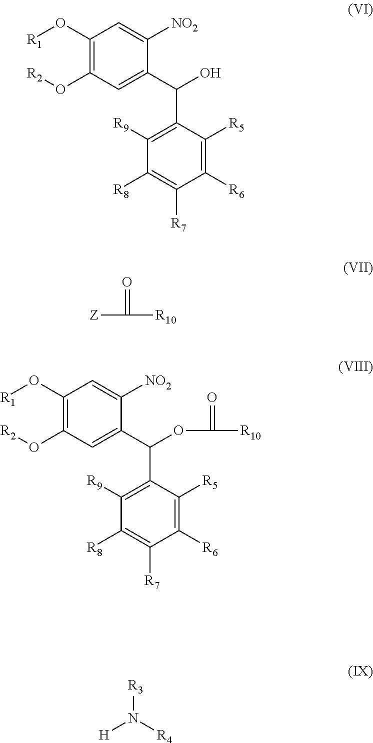
wherein in the general formulae (VI) and (VIII), R₁ and R₂ are each the same as R₁ and R₂ in the general formula (I), and R₅ to R₉ are each the same as R₅ to R₉ in the general formula (I);
in the general formula (VII), Z is a chlorine atom, a bromine atom, an iodine atom, a trichloromethoxy group or a 1-imidazolyl group;
in the general formulae (VII) and (VIII), R₁₀ is a chlorine atom, trichloromethoxy group, 1-imidazolyl group, phenoxy group, 4-nitrophenoxy group or 4-cyanophenoxy group; and
in the general formula (IX), R₃ and R₄ are each the same as R₃ and R₄ in the general formula (I).

11. The process for producing the N-(α-aromatic group-substituted-2-nitro-4,5-dialkoxybenzyloxycarbonyl)amine compound according to claim 10, wherein the compound represented by the general formula (VII) is phosgene, trichloromethyl chloroformate, triphosgene, carbonyl diimidazole, p-nitrophenyl chloroformate or p-cyanophenyl chloroformate.

12. The N-(α-aromatic group-substituted-2-nitro-4,5-dialkoxybenzyloxycarbonyl)amine compound according to claim 2, wherein in the general formula (I), R₁ and R₂ are methyl groups, R₃ is a hydrogen atom, R₄ is a cyclohexyl group and R₅ to R₉ are all hydrogen atoms.
